(19) United States
(12) Patent Application Publication
(10) Pub. No.: US 2013/0156649 A1
(43) Pub. Date: Jun. 20, 2013

(54) PURIFICATION UNIT AND DEODORIDING DEVICE

(71) Applicant: SANYO Electric Co., Ltd., Osaka (JP)

(72) Inventors: Morio NAKATANI, Ichinomiya-shi (JP); Keiichi KURAMOTO, Osaka (JP)

(73) Assignee: SANYO ELECTRIC CO., LTD., Osaka (JP)

(21) Appl. No.: 13/767,908

(22) Filed: Feb. 15, 2013

Related U.S. Application Data
(63) Continuation of application No. PCT/JP2011/061494, filed on May 19, 2011.

(30) Foreign Application Priority Data
Aug. 20, 2010 (JP) 2010-185226

(51) Int. Cl. A61L 9/00

(52) U.S. Cl. CPC A61L 9/00
USPC 422/122

(57) ABSTRACT

A purification unit is provided with a light source which emits light, a purification plate which causes a photocatalysis reaction by irradiation with the light, and a reflection plate which has a curved surface configuration, and is configured to reflect the light emitted from the light source for guiding the reflected light to the purification plate. The layout of the light source and the curved surface configuration of the reflection plate are defined in such a manner that an intensity of the light to be applied to the purification plate is biased toward an upstream side of an air flow path in the purification unit.

This application claims priority under 35 U.S.C. Section 119 of Japanese Patent Application No. 2010-185226 filed on Aug. 20, 2010, entitled “PURIFICATION UNIT AND DEODORIDING DEVICE.” The disclosure of the above application is incorporated herein by reference.

### BACKGROUND OF THE INVENTION

1. Field of the Invention

The present invention relates to a purification unit for purifying a material to be purified that is contained in the air, with use of a photocatalytic structural member, and a deodorizing device.

2. Disclosure of Related Art

In recent years, development has progressed on photocatalytic devices which perform air purification, deodorization, water purification, antibacterial treatment, soil release, water decomposition, with use of a photocatalytic structural member containing a photocatalytically active material. A photocatalytic structural member has such a property that irradiation of light of a predetermined wavelength causes an oxidation-reduction reaction (photocatalysis reaction) on a film surface for purifying a material adhered to the film surface. Generally, the photocatalytic structural member of this kind is formed by laminating a photocatalysis film composed of titanium oxide (TiO₂) or the like on a substrate.

In a purification unit and a deodorizing device incorporated with such a photocatalytic structural member, the air in the vicinity of the device is drawn in through an air intake port, a material to be purified that is contained in the drawn air is purified on the photocatalysis film, and the air after the purification is drawn out through an air exhaust port. In the above configuration, for instance, plural light sources are used for efficiently causing a photocatalysis reaction.

An increase in the number of light sources to be used, however, may increase the cost, and may complicate the control of the light sources.

### SUMMARY OF THE INVENTION

A first aspect according to the invention is directed to a purification unit for purifying air by a photocatalysis reaction. The purification unit according to the first aspect is provided with a light source which emits light, a purification plate which causes the photocatalysis reaction by irradiation of the light, and a reflection plate which has a curved surface configuration and is configured to reflect the light emitted from the light source for guiding the reflected light to the purification plate. In the above configuration, the layout of the light source and the curved surface configuration of the reflection plate are defined in such a manner that an irradiation intensity of the light on to the purification plate is biased toward an upstream side of an air flow path in the purification unit.

A second aspect according to the invention is directed to a deodorizing device. The deodorizing device according to the second aspect is provided with the purification unit according to the first aspect, a fan which causes the air to flow into the deodorizing device, and a controller which controls the fan and the light source in the purification unit.

### BRIEF DESCRIPTION OF THE DRAWINGS

These and other objects, and novel features of the present invention will become more apparent upon reading the following detailed description of the embodiment along with the accompanying drawings.

FIGS. 1A through 1C are diagrams showing a configuration of a purification plate in an embodiment, wherein FIG. 1A is a diagram showing a laminate structure of the purification plate, FIG. 1B is a diagram showing a concave-convex structure of a substrate of the purification plate, and FIG. 1C is a diagram showing a secondary electrophotographic image of the concave-convex structure.

FIG. 2 is a diagram showing a sequence of forming a substrate in the embodiment.

FIGS. 3A through 3D are diagrams for describing a configuration example of a purification unit in the embodiment, wherein FIG. 3A is a schematic diagram showing a reflection efficiency in the case where light enters layers having different refractive indexes, FIG. 3B is a diagram showing a relationship between an incident angle of light which enters a lower surface of a middle layer, and a ratio of light loss on the lower surface of the middle layer, in FIG. 3A, FIG. 3C is a schematic diagram showing a comparative example of a purification unit, in which light sources are disposed in such a manner as to suppress the incident angle of light which enters purification plates C10, and FIG. 3D is a diagram showing a configuration example of a purification unit capable of solving a problem residing in the comparative example.

FIG. 4 is an exploded perspective view of a purification unit in an example.

FIG. 5 is a perspective view showing a configuration of a purification plate of the purification unit in the example.

FIG. 6 is a perspective view of the purification unit in the example.

FIG. 7A is a diagram showing a configuration example (simulation condition) of a purification unit in a first modification, and FIGS. 7B and 7C are diagrams showing simulation results on the purification unit in the first modification.

FIG. 8A is a diagram showing a configuration example (simulation condition) of a purification unit in a second modification, and FIGS. 8B and 8C are diagrams showing simulation results on the purification unit in the second modification.

FIG. 9 is a perspective view of the purification unit in the second modification.

FIG. 10 is a diagram showing a configuration of a deodorizing device in an example.

FIGS. 11A through 11C are diagrams showing a control method to be performed by a control circuit of the deodorizing device in the example, wherein FIG. 11A is a flowchart showing mode switching of the deodorizing device, FIG. 11B is a diagram showing an on-off control pattern when the deodorizing device is in “ON/OFF mode”, and FIG. 11C is a diagram showing an on-off control pattern when the deodorizing device is in “ON mode”.

FIG. 12 is a side view of a purification unit in another modification, when viewed from Y-axis direction.

The drawings are provided mainly for describing the present invention, and do not limit the scope of the present invention.

### DESCRIPTION OF PREFERRED EMBODIMENTS

In the following, an embodiment of the invention is described referring to the drawings.

<Arrangement Example of Purification Plate>

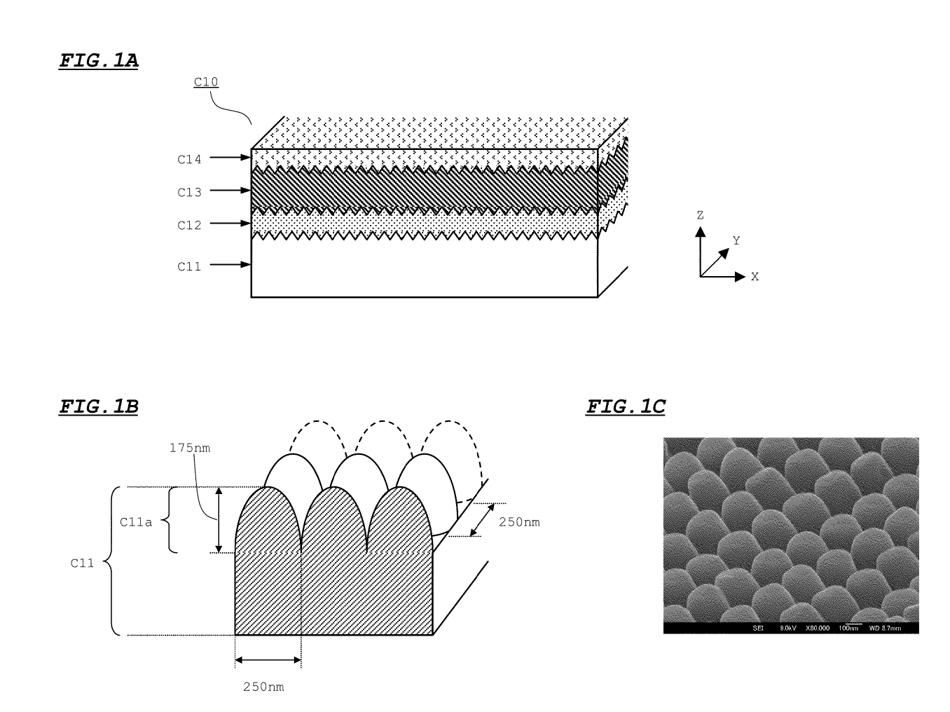

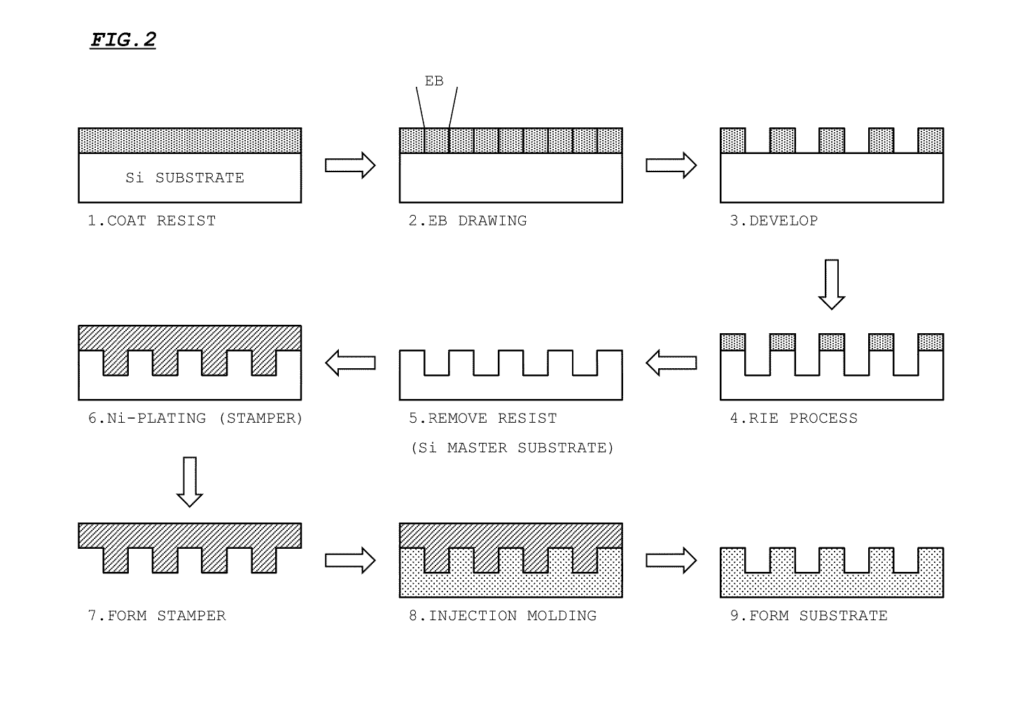

In this section, an arrangement example of a purification plate which causes a photocatalysis reaction by irradiation of light is described referring to FIGS. 1A through 1C and FIG. 2.

FIG. 1A is a diagram showing a laminate structure of a purification plate C10, FIG. 1B is a diagram showing a concave-convex structure C11a of a substrate C11 of the purification plate C10, and FIG. 1C is a diagram showing a secondary electrophotographic image of the concave-convex structure C11a.

Referring to FIG. 1A, the purification plate C10 has the substrate C11, a transmissive film C12, a photocatalysis film C13, and an adsorption film C14.

The substrate C11 is made of a light-transmissive material such as polycarbonate, and the refractive index of the substrate C11 is set to 1.6. As shown in FIGS. 1B, 1C, the concave-convex structure C11a is formed on the transmissive film C12 side surface of the substrate C11 in such a manner that columnar-shaped protrusions are uniformly arranged at a constant pitch in a matrix. The pitch (width of the columnar-shaped protrusion) of the concave-convex structure C11a is 250 nm in length and breadth, and the height of the columnar-shaped protrusion is 175 nm.

The photographic image shown in FIG. 1C is obtained by forming an alloy film of 20 nm on the concave-convex structure C11a by sputtering, followed by image capturing in a state that Pt—Pd is vapor-deposited by 10 Å for electrophotography.

In the following, a sequence of forming the substrate C11 is described referring to FIG. 2.

First, a resist is coated on a silicon master by spin-coating (step 1). Then, a concave-convex structure having the above pitch is formed by EB drawing (electron-beam cutting) (step 2). After the drawing, a developing process is performed (step 3), and then, an RIE process is performed (step 4). Further, the remaining resist is removed by oxygen-plasma asking (step 5). By performing the above steps, a concave-convex structure is formed on the silicon master (Si-master substrate).

Subsequently, the Si master substrate undergoes Ni-plating (step 6) for depositing Ni. Then, a stamper is fabricated by peeling off the deposited Ni-layer from the Si master substrate (step 7). Then, the stamper is subjected to an injection molding process (step 8) for fabricating a substrate C11 (step 9). By performing the above steps, the substrate C11 having a concave-convex structure transferred thereon is formed.

In this embodiment, a light-transmissive material such as polyolefin may be used as the material for the substrate C11, instead of polycarbonate. Further, it is possible to use a biodegradable material such as polylactate, instead of the above. Use of a biodegradable material is advantageous in reducing the environmental load at the time of disposal, for example.

Alternatively, laser beam cutting may be applied, in place of EB drawing. In the modification, a photoresist layer is coated on the silicon master. Further, laser light of or about 400 nm wavelength is used as a cutting beam.

Referring back to FIG. 1A, the transmissive film C12 is laminated on the concave-convex structure C11a of the substrate C11 formed by the aforementioned sequence by a sputtering method. The transmissive film C12 is made of Al₂O₃, and the refractive index of the transmissive film C12 is set to 1.6 so that the refractive index of the transmissive film C12 is substantially equal to the refractive index of the substrate C11. Further, the upper surface and the lower surface of the transmissive film C12 are formed into a concave-convex structure reflecting the concave-convex structure C11a of the substrate C11. Since the transmissive film C12 is made of a non-electrolytic inorganic material, the transmissive film C12 is free from corrosion by a photocatalysis reaction on the photocatalysis film C13 to be described later. Further, since the refractive index of the transmissive film C12 and the refractive index of the substrate C11 are substantially equal to each other, there is an advantage that reflection on interface resulting from a refractive index difference is less likely to occur.

In this embodiment, the film thickness and the Ra (surface roughness) of the transmissive film C12 are set to such values that the substrate C11 is not corroded by the photocatalysis film C13. Further, the film thickness and the Ra of the transmissive film C12 are set to such values that light to be entered from the substrate C11 side sufficiently impinges the photocatalysis film C13, and that light to be entered from the photocatalysis film C13 sufficiently impinges the substrate C11. The control on the Ra of the transmissive film C12 is performed by adjusting the gas pressure at the time of sputtering.

The photocatalysis film C13 is laminated on the upper surface of the transmissive film C12 by a sputtering method. The photocatalysis film C13 is made of TiO₂, and the refractive index thereof is set to 2.5. Further, a concave-convex structure reflecting the concave-convex structure formed on the upper surface of the transmissive film C12 is formed on the upper surface and the lower surface of the photocatalysis film C13. With this arrangement, a structure reflecting the concave-convex structure C11a on the surface of the substrate C11 is formed on the upper surface (the adsorption film C14 side surface) of the photocatalysis film C13. Thus, the surface area of the upper surface of the photocatalysis film C13 increases, and a photocatalysis reaction is likely to occur. Further, since these concave-convex structures have a pitch shorter than the wavelength of light to be irradiated, an apparent refractive index on interface gradually changes in depth direction, which is advantageous in suppressing reflection.

The surface of the photocatalysis film C13 itself after film formation can be made porous by adjusting the gas pressure in laminating. By performing the above operation, the photocatalysis film C13 itself becomes porous, which makes it possible to increase the surface area of the photocatalysis film C13. Further, the surface area of the photocatalysis film C13 can be increased by the concave-convex structure C11a of the substrate C11. If the film thickness of the photocatalysis film C13 is small, it is impossible or difficult to completely cover the upper surface of the transmissive film C12 by the photocatalysis film C13. On the other hand, if the film thickness of the photocatalysis film C13 is large, the concave-convex structure formed on the upper surface of the transmissive film C12 is not reflected on the upper surface (the adsorption film C14 side surface) of the photocatalysis film C13. In addition to the above, if the film thickness of the photocatalysis film C13 is large, light to be entered from the transmissive film C12 side and from the adsorption film C14 side may be absorbed on the photocatalysis film C13, which makes it difficult to transmit the light through the upper surface and the lower surface of the photocatalysis film C13. In view of the above, in this embodiment, the film thickness of the photocatalysis film C13 is set to such a value that the upper surface of the transmissive film C12 is sufficiently covered, and that a sufficient amount of light is transmitted through the photocatalysis film C13.

TiO₂ forming the photocatalysis film C13 contains anatase crystal particles. Anatase crystal absorbs ultraviolet light of 388 nm or smaller in wavelength from a band gap material, and causes a photocatalysis reaction. Further, since anatase crystal exists in the photocatalysis film C13 in the form of particles, the anatase crystal is uniformly distributed in the substrate C11, no matter how intricate the shape of the substrate C11 is. This makes it easy to cause a photocatalysis reaction in a wide range over the photocatalysis film C13 with enhanced efficiency.

Further, it is known that TiO₂ forms a rutile structure, an amorphous structure, a brookite structure, other than the anatase crystal structure, and the photocatalysis reaction differs depending on the structure. Specifically, the reaction activity and the reaction wavelength differ in each of the structures. TiO₂ forming the photocatalysis film C13 contains plural types of structures.

Specifically, the photocatalysis film C13 composed of TiO₂ has an anatase crystal structure, and is a composite film containing, in addition to the anatase crystal structure, defects in amorphous structure or anatase crystal structure, particles containing a trace of nitrogen at the time of sputtering, and rutile particles. By the containment of these matters, the photocatalysis reaction on the photocatalysis film C13 progresses not only by the light at a wavelength of 388 nm or shorter as described above, but also by the light at a wavelength in a visible light region in the range of from 400 to 500 nm. In view of the above, in the case where an LED or a semiconductor laser is used as a light source for causing a photocatalysis reaction, even if the light to be emitted from these light sources contains visible light (light at a wavelength of 388 nm or longer) due to a temperature difference or an individual difference among the light sources, it is possible to enhance the light use efficiency. It should be noted that a light source such as an LED or a semiconductor laser light source has such a characteristic that as the wavelength of light to be outputted is shortened, the manufacturing cost increases, and as the wavelength of light to be outputted is closer to a visible light wavelength, it is possible to manufacture the light source at a lower cost. In view of the above, in the case where a light source for outputting ultraviolet light is used as a light source, it is not necessary to include all the aforementioned structures except for the anatase crystal structure, as the structures to be formed of TiO₂. As far as a composite film contains particles capable of obtaining photocatalytic activity by irradiation with visible light (light at a wavelength of 388 nm or longer), any composite film may be used as the photocatalysis film C13 composed of TiO₂. The photocatalysis film C13 may be a film composed of a perfect anatase crystal structure. In the above case, however, photocatalytic activity is obtained only at a wavelength of 388 nm or shorter. Thus, it is necessary to strictly select a light source for efficiently obtaining photocatalytic activity, which makes it impossible to configure a light source at a low cost.

The photocatalysis film C13 photocatalytically acts on a material adhered to the photocatalysis film C13. Examples of a material which undergoes a photocatalytic action include ammonium, acetaldehyde, hydrogen sulfide, methyl mercaptan, formaldehyde, acetic acid, toluene, bacteria and oils. These materials undergo a photocatalytic action and are decomposed into carbon dioxide, water, and the like.

The adsorption film C14 is laminated on the upper surface of the photocatalysis film C13 by a sputtering method. The adsorption film C14 is composed of SiO₂ having light transmissivity, and the refractive index thereof is 1.45. SiO₂ has moisture absorbency, and has a property that it is likely to bind with water molecules in the air or a vapor phase gas. With use of the adsorption film C14 having the above property, the material in the air that exists on the upper surface of the adsorption film C14 is easily adhered to the adsorption film C14. Further, the material adsorbed onto the adsorption film C14 is trapped on the adsorption film C14 and is likely to undergo a photocatalytic action by the adsorption film C14.

The adsorption film C14 is laminated on the photocatalysis film C13 in such a manner that the upper surface of the photocatalysis film C13 is not completely coated. Further, if the adsorption film C14 is formed to have such a thickness that reflects the concave-convex structure on the photocatalysis film C13, the refractive index gradually changes in depth direction, because the pitch of the concave-convex structure of the adsorption film C14 is shorter than the wavelength of light. With this arrangement, it is less likely to cause reflection on the adsorption film C14, which makes it easy to transmit the light through the adsorption film C14. Further, forming a concave-convex structure on the photocatalysis film C13 is advantageous in increasing the adsorption rate resulting from an increase in the surface area. In this case, it is more preferable that the adsorption film C14 has a porous structure. Specifically, multitudes of micropores are formed in the adsorption film C14 by lowering the gas pressure at the time of sputtering (specifically, 0.8 through 1 Pa or higher), or increasing the sputtering rate (70 Å/min or larger). By performing the above operation, the material adhered to the upper surface of the adsorption film C14 is contacted with the photocatalysis film C13 through the micropores. Further, the above arrangement allows the light to be entered to the adsorption film C14 to transmit through the adsorption film C14 so that the light is easily transmitted through the photocatalysis film C13. It is desirable to set the film thickness of the adsorption film C14 to such a value that the material adhered to the adsorption film C14 is efficiently contacted with the photocatalysis film C13 so that light is easily transmitted.

In the case where ultraviolet light at a wavelength of 375 nm is applied to the thus-configured purification plate C10 from the lower surface side of the substrate C11 or from the upper surface side of the adsorption film C14, such ultraviolet light impinges onto the photocatalysis film C13. Then, the material coming from the adsorption film C14 side and in contact with the photocatalysis film C13 is allowed to undergo a photocatalytic action.

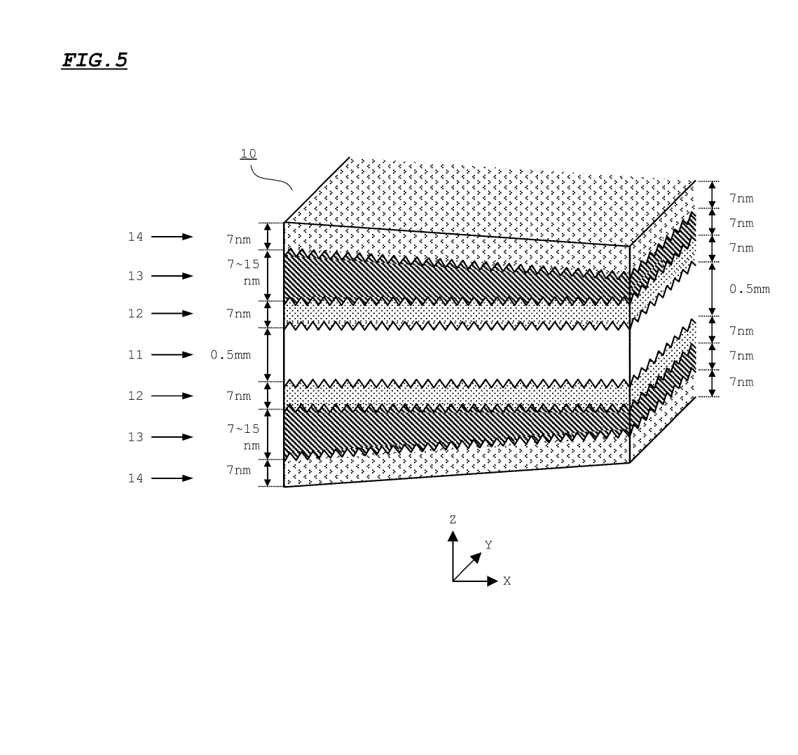

It may be possible to laminate the transmissive film C12, the photocatalysis film C13, and the adsorption film C14 shown in FIG. 1A on the lower surface of the purification plate C10, as well as on the upper surface of the purification plate C10. The above modification is advantageous in enhancing the purification performance by one purification plate C10. As will be described later in the section of “Example of Purification Unit”, as shown in FIG. 5, a purification plate 10 is configured to vary the thickness of a photocatalysis film 13 in X-axis direction of FIG. 5. This will be described later in detail.

<Configuration Example of Purification Unit>

In this section, a configuration example of a purification unit incorporated with the purification plates C10 is described.

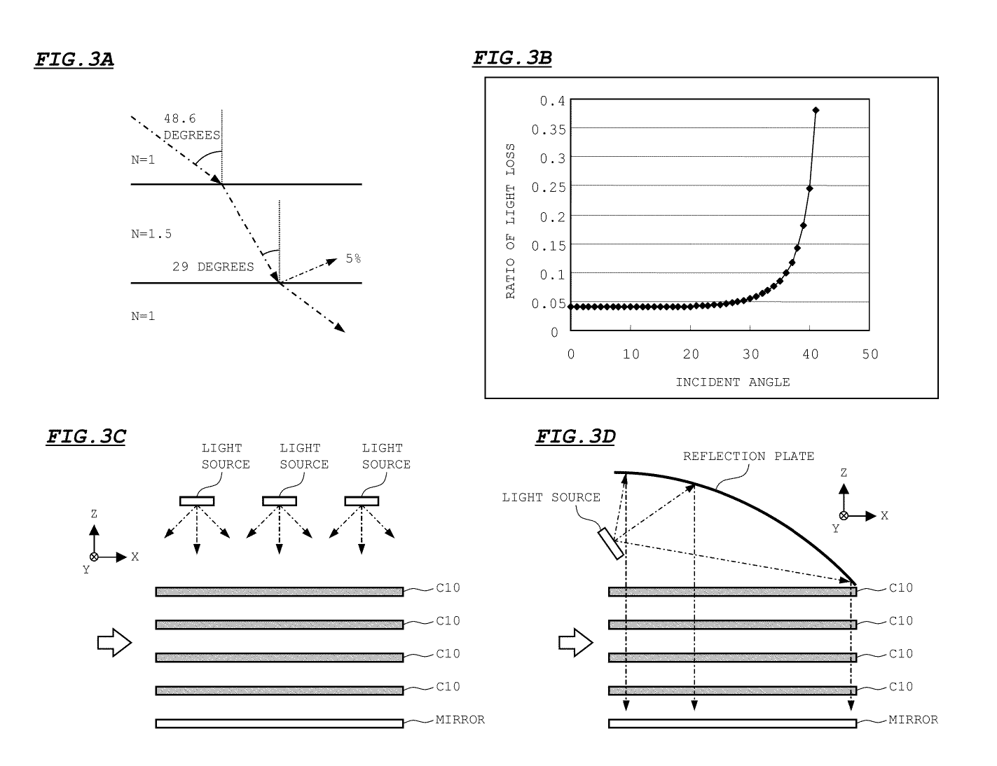

FIG. 3A is a schematic diagram showing a reflection efficiency in the case where light enters layers having different refractive indexes.

As shown in FIG. 3A, a layer (middle layer) having a refractive index n of 1.5 is disposed in a medium (e.g. air) having a refractive index n of 1. In the case where the incident angle of light which enters the upper surface of the middle layer is set to 48.6 degrees, the incident angle of the light which enters the lower surface of the middle layer is 29 degrees. In this configuration, 5% of light which enters the lower surface of the middle layer is reflected on the lower surface of the middle layer. In other words, 5% of light which enters the upper surface of the middle layer at an incident angle of 48.6 degrees is lost on the lower surface of the middle layer.

FIG. 3B is a diagram showing a relationship between an incident angle of light which enters the lower surface of the middle layer, and a ratio of light loss on the lower surface of the middle layer, in FIG. 3A.

As shown in FIG. 3B, in the case where the incident angle of light which enters the lower surface of the middle layer is about 29 degrees or smaller, the ratio of light loss is substantially constant (5%). However, in the case where the incident angle exceeds about 29 degrees, the ratio of light loss is drastically increased over 5%. This shows the following. Specifically, in the case where light is allowed to enter from an air layer into a medium (e.g. the middle layer shown in FIG. 3A) having a refractive index larger than 1, it is desirable to minimize the incident angle of light which enters the medium to thereby minimize the incident angle of light with respect to the lower surface of the medium in the aspect of suppressing the light loss.

In this example, let us assume a case that a purification unit is incorporated with purification plates C10, for instance, a plurality of the purification plates C10 as exemplified in FIG. 1A are laminated in a vertical direction (Z-axis direction), and light is applied to the purification plates C10 from above the uppermost purification plate C10. In this configuration, as is obvious from the results shown in FIGS. 3A and 3B, it is desirable to suppress the incident angle of light which enters the purification plate C10 as much as possible for efficiently causing a photocatalytic action on the purification plate C10. Specifically, in FIG. 3A, it is desirable to set the incident angle of light with respect to the upper surface of the middle layer to 48.6 degrees or smaller.

FIG. 3C is a schematic diagram showing a comparative example of a purification unit, in which light sources are disposed in such a manner as to suppress the incident angle of light which enters purification plates C10 as much as possible.

As shown in FIG. 3C, four purification plates C10 are laminated one over the other with a certain clearance in a vertical direction (Z-axis direction). A mirror is disposed below the purification plate C10 farthest from the light sources. The air containing a material to be purified is fed from the left side of the purification plates C10 to the right side of the purification plates C10 through the clearances formed between the purification plates C10 in the vertical direction.

The three light sources are disposed away from each other in X-axis direction at a predetermined interval, above the purification plate C10 closest to the light sources, in accordance with the X-axis directional width of the purification plates C10. It should be noted that plural light sources (not shown) are disposed in Y-axis direction of each of the three light sources in accordance with the Y-axis directional width of the purification plates C10. Light (e.g. light from an LED or a semiconductor laser) having a small spread angle is emitted from the light sources. Each of the light sources is disposed in such a manner that the optical axis of light to be emitted from each light source perpendicularly intersects the purification plates C10. Since the light to be emitted from each of the light sources has a small spread angle, the plural light sources are disposed as described above, so that light is applied to the entirety of the surfaces of the purification plates C10.

In the case where the purification plates C10 and the light sources are disposed as described above, light emitted from each of the light sources is transmitted through the four purification plates C10, and is reflected on the mirror. Light reflected on the mirror travels upwardly and enters the four purification plates C10 again. By performing the above operation, the material to be purified that is in contact with the photocatalysis film C13 of each of the purification plates C10 is purified by a photocatalysis reaction.

However, in the case where the light sources are disposed in the purification unit as shown in FIG. 3C, it is necessary to dispose the plural light sources in X-axis direction in accordance with the X-axis directional length of the purification plates C10. This may increase the cost, and may complicate the control of the light sources.

FIG. 3D is a diagram showing a configuration example of a purification unit capable of solving the above problem.

In the configuration shown in FIG. 3D, only one light source is disposed in X-axis direction, and a reflection plate having a curved surface configuration is disposed above the purification plate C10 closest to the light source. It should be noted that plural light sources (not shown) are disposed in Y-axis direction of the one light source in accordance with the Y-axis directional width of the purification plates C10.

In the above configuration, the light source is inclined with a predetermined angle with respect to Y-axis, so that a center axis of a light flux from the light source enters a position displaced toward X-axis minus side with respect to an X-axis directional center of the reflection plate. In this configuration, a portion of light having a largest intensity and to be emitted from the light source enters the position displaced toward X-axis minus side with respect to the X-axis directional center of the reflection plate. Further, the reflection plate has a parabolic curve when viewed from Y-axis direction, and the light source is disposed at a focal point of the parabolic curve in X-Z plane.

In the case where the light source and the reflection plate are disposed as described above, light emitted from the light source is reflected at any position on the reflection plate in Z-axis minus direction by the reflection plate, and enters the purification plates C10 in a direction perpendicular thereto. This avoids oblique incidence of light onto the purification plates C10. Thus, the above configuration is more advantageous in suppressing the light loss, as compared with the configuration shown in FIG. 3C.

Further, only one light source is disposed in X-axis direction, and it is possible to irradiate a wide area on the purification plates C10 by the one light source. Accordingly, the above configuration is advantageous in suppressing the cost, and in simplifying the control of the light source, as compared with the configuration shown in FIG. 3C.

Further, in the case where the light source and the reflection plate are disposed as described above, the density of light rays reflected on the reflection plate increases, as the light travels in X-axis minus direction, based on the layout relationship between the light source and the reflection plate. As a result, as shown in FIG. 3D, the intensity distribution of light on the purification plates C10 is such that the distribution is biased toward X-axis minus side in the irradiation area on the purification plates C10. Furthermore, the light source is inclined in such a manner that a portion of light having a large light intensity enters a region on the purification plates C10 corresponding to X-axis minus side with respect to the X-axis directional center of the purification plates C10. Accordingly, the irradiation position of light having a largest light intensity on the purification plates C10 is shifted toward X-axis minus side with respect to the X-axis directional center of the purification plates C10. In this configuration, the purification performance of a purification unit 100 is enhanced on the air intake side, in other words, on X-axis minus side of the purification plates C10. Accordingly, the above configuration is advantageous in promptly promoting the purification action, even in the case where the drawn air contains a large amount of a material to be purified.

Further, the light intensity on X-axis plus side of the purification plates C10 is weakened, as compared with X-axis minus side. Accordingly, it is possible to suppress a temperature rise by light absorption on X-axis plus side of the purification plates C10. This enables to purify the air on X-axis minus side of the purification plates C10, and enables to easily adhere the material to be purified on X-axis plus side of the purification plates C10, even in the case where the air does not contain a large amount of the material to be purified. Accordingly, the above configuration is advantageous in efficiently promoting the purification action.

In the configuration shown in FIG. 3D, in the case where light from the light source is directed further to the left side than an upper end of the reflection plate, a mirror for directing the light toward the reflection plate may be provided above the light source.

<Example of Purification Unit>

In this section, an example of a purification unit based on the layout example of a light source and a reflection plate shown in FIG. 3D is described.

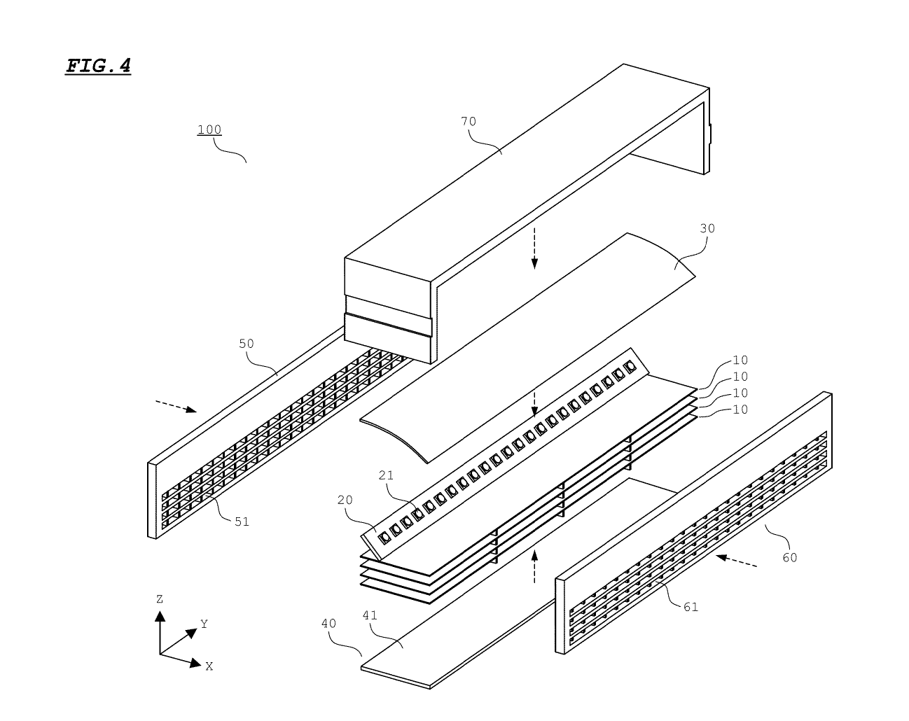

FIG. 4 is an exploded perspective view of a purification unit 100 in the example.

The purification unit 100 is provided with four purification plates 10, a light emitting unit 20, a reflection plate 30 having a curved surface configuration, a base member 40, a front plate 50, and a back plate 60. It should be noted that the purification plates 10, the light emitting unit 20 (LED 21), and the reflection plate 30 and the base member 40 (mirror 41) shown in FIG. 4 respectively correspond to the purification plates C10, the light source, and the reflection plate shown in FIG. 3D.

FIG. 5 is a perspective view showing a configuration of the purification plate 10 to be used in the example. As shown in FIG. 5, the purification plate 10 is configured in such a manner that transmissive films 12, photocatalysis films 13, and adsorption films 14 are laminated in a vertical direction with respect to a substrate 11. Referring to FIG. 5, to simplify the description, the upper surface of the upper-side adsorption film 14, and the lower surface of the lower-side adsorption film 14 are each illustrated as a flat surface. Actually, however, these surfaces are formed into a concave-convex structure reflecting the concave-convex structure of the substrate 11.

As shown in FIG. 5, the photocatalysis film 13 is configured to have a thickness on X-axis minus side larger than that on X-axis plus side. In the case where the photocatalysis film 13 having a varied thickness in X-axis direction as described above is laminated, sputtering is performed by inclining a structure in which the transmissive films 12 are laminated on the substrate 11, with respect to an axis of rotation of a sputtering device. By performing the above operation, the photocatalysis films 13 are laminated with a varied thickness depending on the distance from the axis of rotation.

The thickness of each layer is set as shown in e.g. FIG. 5. Specifically, the thickness of the substrate 11 is set to 0.5 mm, the thickness of the transmissive film 12 is set to 7 nm, the thickness of the photocatalysis film 13 on an X-axis plus end thereof is set to 7 nm, the thickness of the photocatalysis film 13 on an X-axis minus end thereof is set in the range of from 7 to 15 nm, and the thickness of the adsorption film 14 is set to 7 nm.

In the case where the thickness of the photocatalysis film 13 on X-axis minus side is set to be large as described above, the purification performance of the photocatalysis film 13 increases on X-axis minus side. Specifically, because the photocatalysis film 13 is porous as described above, it is possible to contact the photocatalysis film 13 on X-axis minus side with a larger amount of a material to be purified. Accordingly, it is easy to purify the material to be purified that is contained in the air in the vicinity of the purification plate 10 by contact with the photocatalysis film 13 on X-axis minus side. Thus, configuring the purification plate 10 as described referring to FIG. 5 is advantageous in enhancing the purification performance of the purification plate 10 on X-axis minus side, as compared with X-axis plus side.

Referring back to FIG. 4, as shown in FIG. 4, each of the purification plates 10 has a smaller length in X-axis direction than in Y-axis direction, and the purification plates 10 are laminated at a predetermined interval substantially in the same manner as the purification plates C10 shown in FIG. 3D.

The light emitting unit 20 has twenty-three LEDs 21 in Y-axis direction. The light emitting unit 20 causes each of the LEDs 21 to emit light, based on a control signal to be inputted to the light emitting unit 20. The LED 21 emits light at a wavelength of 375 nm toward the reflection plate 30. The light emitting unit 20 is configured in such a manner that twenty-three LEDs 21 are disposed in Y-axis direction. The invention is not limited to the above. The number of LEDs 21 to be disposed in Y-axis direction may be adjusted, as necessary.

The reflection plate 30 is a mirror having a curved surface configuration, and reflects light emitted from each of the LEDs 21 toward the purification plates 10. A flat plate-shaped mirror 41 is disposed on the upper surface of the base member 40. A reflection film for reflecting light of an irradiation wavelength (375 nm in the example) is formed on each of the reflection plate 30 and the mirror 41. Specifically, the reflection film is made of Ag, or an alloy of Ag and Al, and has a reflectance of 80% or more. Further, the higher the reflectance is, the better the performance of the device is.

Plural vents 51 passing through X-axis direction are formed in the front plate 50 at a position where the purification plates 10 are disposed. Plural vents 61 passing through X-axis direction are formed in the back plate 60 at a position where the purification plates 10 are disposed. Alternatively, an opening may be formed in the front plate 50, and a filter for removing dust and the like may be mounted in the opening, in place of the vents 51 formed in the front plate 50. Likewise, an opening may be formed in the back plate 60, and a filter may be mounted in the opening, in place of the vents 61 formed in the back plate 60. A cover 70 has an upper surface in parallel to X-Y plane, and two side surfaces in parallel to X-Z plane.

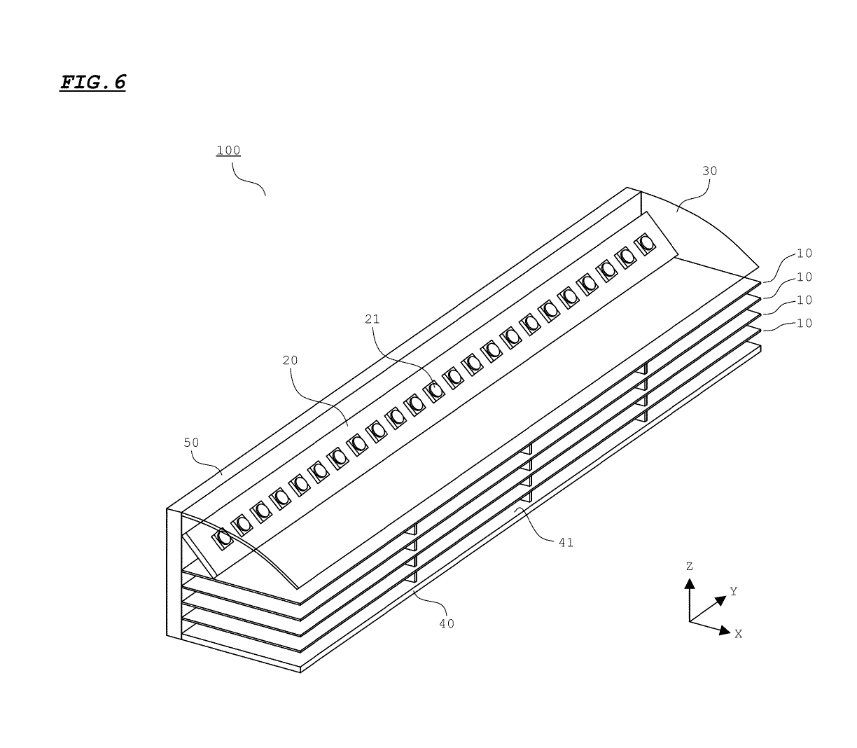

As shown in FIG. 6, by assembling the components shown in FIG. 4, the purification unit 100 is fabricated. Referring to FIG. 6, to simplify the description, illustration of the cover 70 and the back plate 60 is omitted. Further, to simplify the description, the reflection plate 30 is depicted as a transparent plate.

After the purification unit 100 is assembled as described above, and when a stream of air is fed in X-axis plus direction into the purification unit 100 through the vents 51 formed in the front plate 50, the stream of air is guided in X-axis plus direction while passing through the four purification plates 10. As the stream of air passes through the purification plates 10, the material to be purified that is contained in the air is adhered to the adsorption films 14 of the purification plates 10. Specifically, as described above, the material to be purified in the air in the vicinity of the purification plates 10 is stagnated on the adsorption films 14, and is contacted with the photocatalysis films 13. When the light emitted from the LEDs 21 of the light emitting unit 20 is applied to the photocatalysis films 13 in this state, a photocatalysis reaction occurs, and the material to be purified in contact with the photocatalysis films 13 is decomposed. The air purified by decomposition of the material to be purified is drawn out through the vents 61 formed in the back plate 60.

As described above, in the purification unit 100 of the example, light emitted from one and only LED 21 which is disposed in X-axis direction is applied to a wide area on the purification plates 10 by the reflection plate 30 having a curved surface configuration. In this configuration, it is not necessary to dispose plural LEDs in X-axis direction. Accordingly, as compared with a configuration, in which plural LEDs are disposed in X-axis direction, the above configuration is advantageous in suppressing a cost increase, and in simplifying the control of an LED. Further, light reflected on the reflection plate 30 enters the purification plates 10 in a direction substantially perpendicular thereto. Accordingly, it is possible to suppress the ratio of light loss resulting from oblique incidence of light onto the purification plates 10.

It is desirable to irradiate the reflection plate 30 with light emitted from a center of the LED 21 in order to apply a large amount of light to the reflection plate 30 in a direction substantially perpendicular thereto. Specifically, it is desirable to irradiate the reflection plate 30 with 50% or more of light to be emitted from the LED 21. Irradiating the reflection plate 30 with an amount of light as much as possible enables to reflect a larger amount of light on the reflection plate 30 for irradiating the photocatalysis films 13.

Further, in the purification unit 100 of the example, as described referring to FIG. 3D, light emitted from the LED 21 is applied with a larger intensity to X-axis minus side of the purification plates 10 than X-axis plus side of the purification plates 10. Further, as described referring to FIG. 5, the photocatalysis films 13 of the purification plates 10 are configured to have a larger thickness on X-axis minus side than X-axis plus side. The above configuration enables to enhance the purification performance of the purification unit 100 on the air intake side i.e. on the front plate 50 side. This is advantageous in promptly promoting the purification action, even in the case where the air drawn from the front plate 50 side contains a large amount of a material to be purified.

Further, in the purification unit 100 of the example, light emitted from the LED 21 is applied with a weaker intensity to X-axis plus side of the purification plates 10 than X-axis minus side of the purification plates 10. Further, the photocatalysis films 13 of the purification plates 10 are configured to have a smaller thickness on X-axis plus side than X-axis minus side. The above configuration enables to suppress a temperature rise on X-axis plus side of the purification plates 10. Accordingly, even for the air which has been purified in the vicinity of the front plate 50 and does not contain a large amount of the material to be purified, it is possible to easily adhere the material to be purified on X-axis plus side of the purification plates 10. This is advantageous in efficiently promoting the purification action.

<First Modification of Purification Unit>

In the case where the LED 21, the reflection plate 30, the purification plates 10, and the mirror 41 are disposed based on the layout as shown in FIG. 3D, the size of the purification unit 100 may be increased, although the aforementioned advantages are obtained.

In view of the above, the inventor of the present application proposes an idea of adjusting the layout of the LED 21, the reflection plate 30, the purification plates 10, and the mirror 41 in such a manner as to miniaturize the purification unit 100 configured based on FIG. 3D to an insisted size.

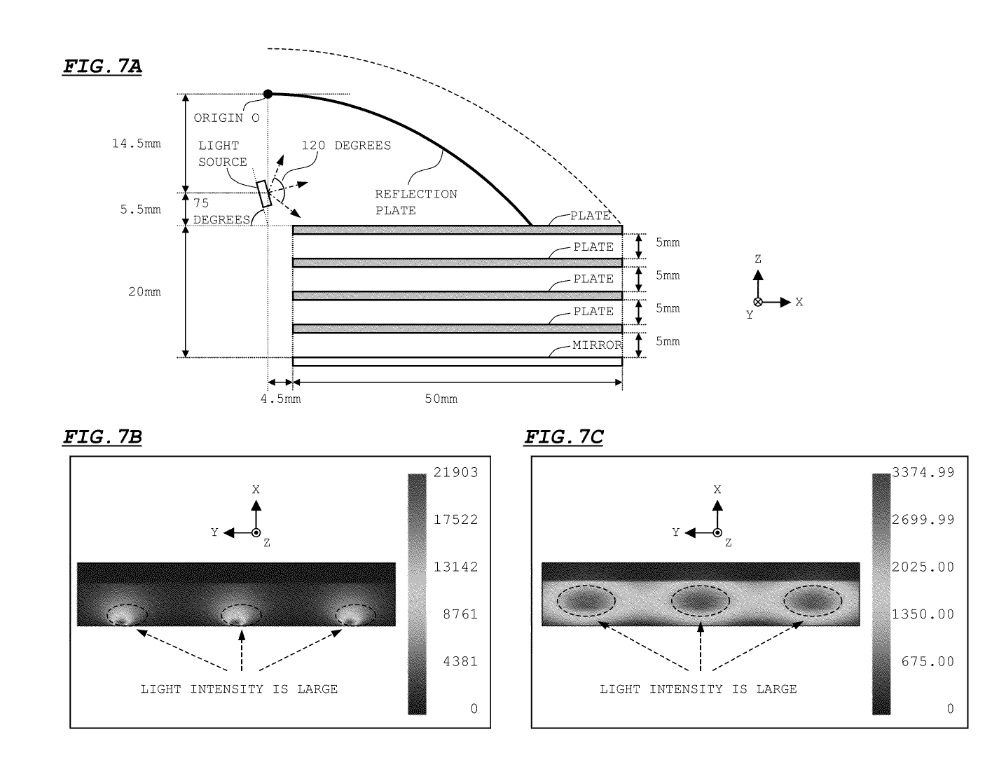

FIGS. 7A through 7C are diagrams respectively showing a configuration and simulation results on a light intensity distribution of the purification plates 10, in the case where the purification unit 100 is miniaturized by adjusting the layout of the LED 21, the reflection plate 30, the purification plates 10, and the mirror 41.

Also, FIG. 7A shows a simulation condition. In the simulation, there are used plates each having a refractive index of 1.5, and a transmittance of 80%, in place of the purification plates 10.

Assuming that the uppermost point of the curved surface configuration of the reflection plate in X-Z plane is the origin O, the curved surface configuration is expressed by a parabolic curve: z=−0.014x². The spread angle of light to be emitted from a light source is 120 degrees, and the light source is inclined at an angle of 75 degrees with respect to the upper surface of each plate. Further, there are disposed three light sources inclined as described above in Y-axis direction. There is a clearance of 0.32725 mm between the right end of the reflection plate and the upper surface of the plate closest to the light sources. The other simulation condition is as shown in FIG. 7A.

In the above configuration, the position of the light sources in a vertical direction (Z-axis direction) is positioned slightly upward from the focal point of the parabolic curve representing the configuration of the reflection plate in X-Z plane. With this configuration, the light use efficiency can be maintained, because the incident angle of light reflected on the reflection plate is close to zero, although the light does not enter the plates in a direction perpendicular thereto.

Further, the light reflected on the reflection plate does not enter the plates in a direction perpendicular thereto, and the light intensity on the plates is shifted slightly toward X-axis plus side. However, since the light emitted from the light sources directly enters a region on the plates near the light sources, it is possible to bias the light intensity on the plates toward X-axis minus side.

Further, in the above configuration, the right end of the reflection plate is located on the left side with respect to the right end of the plates. Accordingly, it is less likely that the light reflected on the reflection plate enters the right-end region on the plates. However, the plates are irradiated with, in addition to the light reflected on the reflection plate, the light which directly enters from the light sources, and the light which directly enters from the light sources and is reflected on the mirror. Accordingly, it is possible to apply light even to the right-end region of the plates.

FIGS. 7B and 7C show simulation results respectively showing an intensity distribution of light which is applied to the uppermost plate closest to the light sources and the lowermost plate farthest from the light sources in the condition shown in FIG. 7A. FIGS. 7B and 7C are diagrams, in which a color image is converted into a monochromatic image.

It is clear from FIGS. 7B and 7C that light spreads and is applied to the plates in X-axis direction. Further, it is clear that the light intensity increases from the center of the plates toward X-axis minus side. In this configuration, even in the case where the purification unit 100 is miniaturized by adjusting the layout of the LED 21, the reflection plate 30, the purification plates 10, and the mirror 41 from the layout shown in FIG. 3D, it is possible to provide the advantages as described in the example of the purification unit 100.

In the layout shown in FIG. 3D, light reflected on the reflection plate travels in Z-axis minus direction. Accordingly, it is necessary to dispose the reflection plate as shown by the broken line in FIG. 7A in order to apply light to the overall X-axis directional width of the plates. In the above configuration, the size of the purification unit 100 may be increased in Z-axis direction as compared with the first modification. Accordingly, the first modification is advantageous in miniaturizing the purification unit 100, as compared with the configuration shown in FIG. 3D.

<Second Modification of Purification Unit>

In the case where it is desired to increase the light intensity on the right-end region of the purification plates 10 from the state that the LED 21, the reflection plate 30, the purification plates 10, and the mirror 41 are disposed based on FIG. 7A, it is possible to dispose three reflection plates, in place of the reflection plate 30.

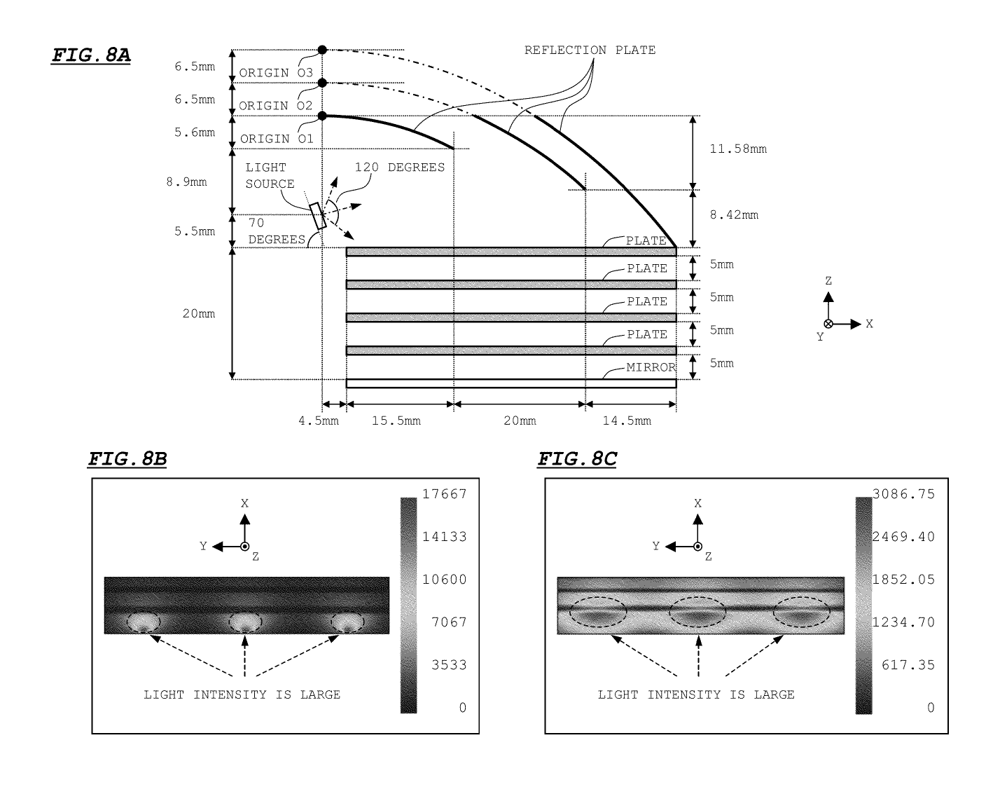

FIGS. 8A through 8C are diagrams respectively showing a configuration and simulation results on the light intensity distribution of purification plates 10, in the case where three reflection plates are disposed.

Also, FIG. 8A shows a simulation condition.

The origins O1 through O3 are respectively uppermost points of curved surface configurations of the left-side reflection plate, the middle reflection plate, and the right-side reflection plate, when viewed from Y-axis direction. The curved surface configurations of the left-side reflection plate, the middle reflection plate, and the right-side reflection plate are respectively expressed by z=−0.014x², z=−0.0113x², and z=−0.011x² in X-Z plane. Further, as shown in FIG. 8A, the three reflection plates are formed only at a portion lower than the position of the origin O1. A light source is inclined at an angle of 70 degrees with respect to the upper surface of the plates. The other simulation condition is as shown in FIG. 8A.

FIGS. 8B and 8C show simulation results respectively showing intensity distributions of light to be applied to the uppermost plate closest to the light sources and the lowermost plate farthest from the light sources in the condition shown in FIG. 8A.

It is clear from FIGS. 8B and 8C that light spreads in X-axis direction and is applied to the plates, and that the light intensity increases from the center of the plates toward X-axis minus side substantially in the same manner as the configuration shown in FIGS. 7B and 7C. Further, it is clear that a large amount of light is applied even to a region on the plates corresponding to X-axis plus side with respect to the center of the plates, as compared with the configuration shown in FIGS. 7B and 7C. Specifically, in the second modification, light which enters from the light sources into the middle reflection plate and into the right-side reflection plate is reflected by these reflection plates and is guided to the plates. Accordingly, the second modification is more advantageous in applying a larger amount of light to the region of the plates corresponding to X-axis plus side, as compared with the configuration shown in FIGS. 7B and 7C.

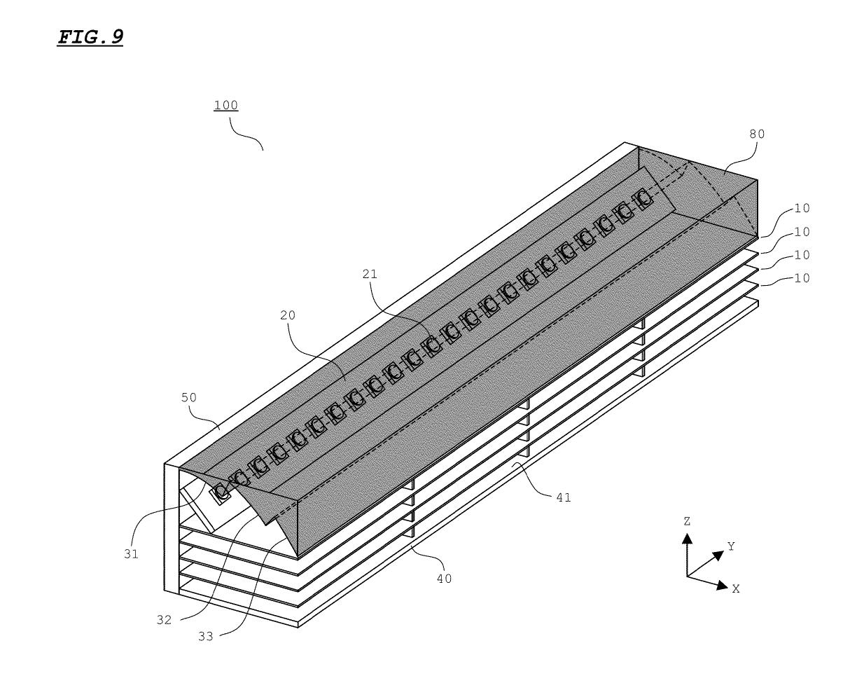

FIG. 9 is a perspective view of a purification unit 100, in the case where three reflection plates are disposed in the purification unit 100, as shown in FIG. 8A. Referring to FIG. 9, to simplify the description, illustration of a cover 70 and a back plate 60 is omitted. Further, reflection plates 31 through 33 are depicted as transparent plates.

As shown in FIG. 9, the reflection plates 31 through 33 corresponding to the three plates shown in FIG. 8A are disposed below an inclined surface formed on the lower surface side of a support block 80. The purification unit 100 as configured above is more advantageous in applying a larger amount of light even to a region on the purification plates 10 corresponding to X-axis plus side, as compared with the purification unit 100 configured based on FIG. 7A. Accordingly, the second modification is advantageous in enhancing the purification performance even on the back plate 60 side of the purification unit 100.

<Example of Deodorizing Device>

The following is an example, in which the aforementioned purification unit 100 is incorporated in a deodorizing device.

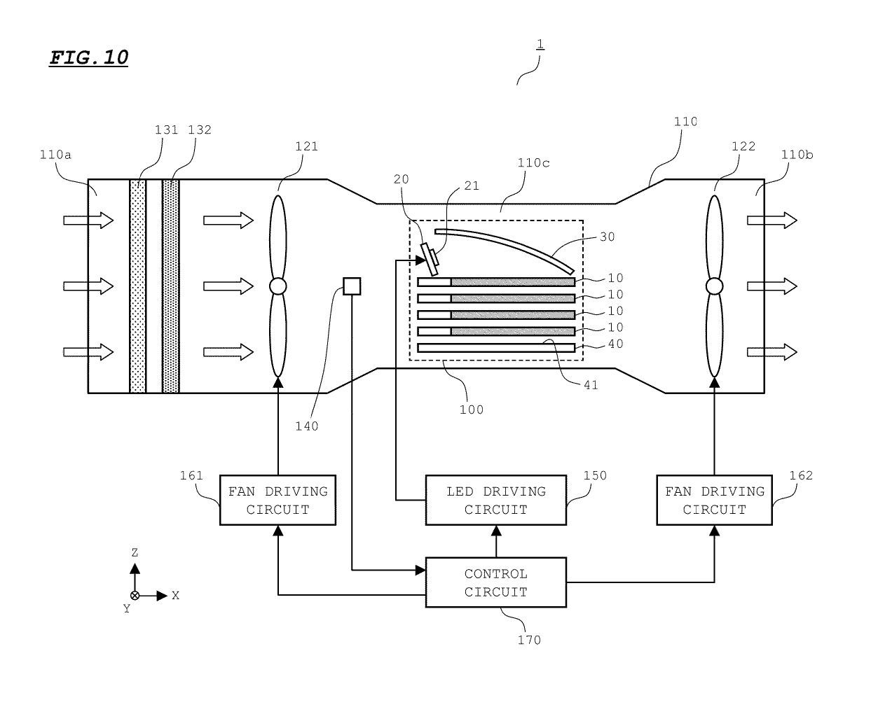

FIG. 10 is a diagram showing a configuration of a deodorizing device 1.

The deodorizing device 1 is provided with a purification unit 100, an air feeding path 110, fans 121, 122, filters 131, 132, an odor sensor 140, an LED driving circuit 150, fan driving circuits 161, 162, and a control circuit 170. The purification unit 100 to be used in the example may be a purification unit configured based on any one of the configurations shown in FIGS. 3D, 7A, and 8A. In FIG. 10, to simplify the description, the purification unit 100 configured based on FIG. 3D is shown.

The air feeding path 110 is formed into a hollow tubular member, and is so configured that the air is allowed to flow in X-axis direction. An entrance and an exit of the air feeding path 110 are respectively formed with an air intake port 110a and an air exhaust port 110b. Further, a purification region 110c for disposing the purification unit 100 is defined near the center of the air feeding path 110.

As described above, the purification unit 100 is provided with four purification plates 10, a light emitting unit 20 having plural LEDs 21 in Y-axis direction, a reflection plate 30 having a curved surface configuration, and a base member 40 carrying a mirror 41 on the upper surface thereof.

The fans 121, 122 cause the air to flow from the air intake port 110a toward the air exhaust port 110b. By performing the above operation, the air in the vicinity of the air intake port 110a is drawn in through the air intake port 110a by the fan 121, passes through the purification region 110c, and is drawn out through the air exhaust port 110b by the fan 122.

The filter 131 removes large dust particles contained in the air that is drawn in through the air intake port 110a, and the filter 132 removes small dust particles contained in the air that is drawn out from the filter 131 side. The odor sensor 140 detects an odor component contained in the air that is fed out toward the purification region 110c by the fan 121. A detection signal from the odor sensor 140 is outputted to the control circuit 170.

The LED driving circuit 150 drives each of the LEDs 21 disposed in the light emitting unit 20 in accordance with an instruction from the control circuit 170. The fan driving circuits 161 and 162 respectively drive the fans 121 and 122 in accordance with an instruction from the control circuit 170. The rotation number of the fans 121 and 122 is controlled by the control circuit 170. The control circuit 170 controls the LED driving circuit 150, and the fan driving circuits 161 and 162, based on an output signal from the odor sensor 140.

In the deodorizing device 1 thus configured, dust in the air drawn in through the air intake port 110a is removed by the filters 131 and 132 by driving the fan 121, and then, the air after the dust removal is fed to the purification region 110c. The air fed to the purification region 110c is drawn into the purification unit 100, and as described above, the material to be purified is decomposed in the purification unit 100.

The air that has been purified in the purification unit 100 is drawn out of the purification unit 100 through the vents 61 (see FIG. 5) formed in the back plate 60, and is fed toward the fan 122. The air in the purification region 110c is fed toward the air exhaust port 110b, and is fed out of the purification unit 100 through the air exhaust port 110b by driving the fans 121 and 122. In this way, the air in the vicinity of the deodorizing device 1 is purified.

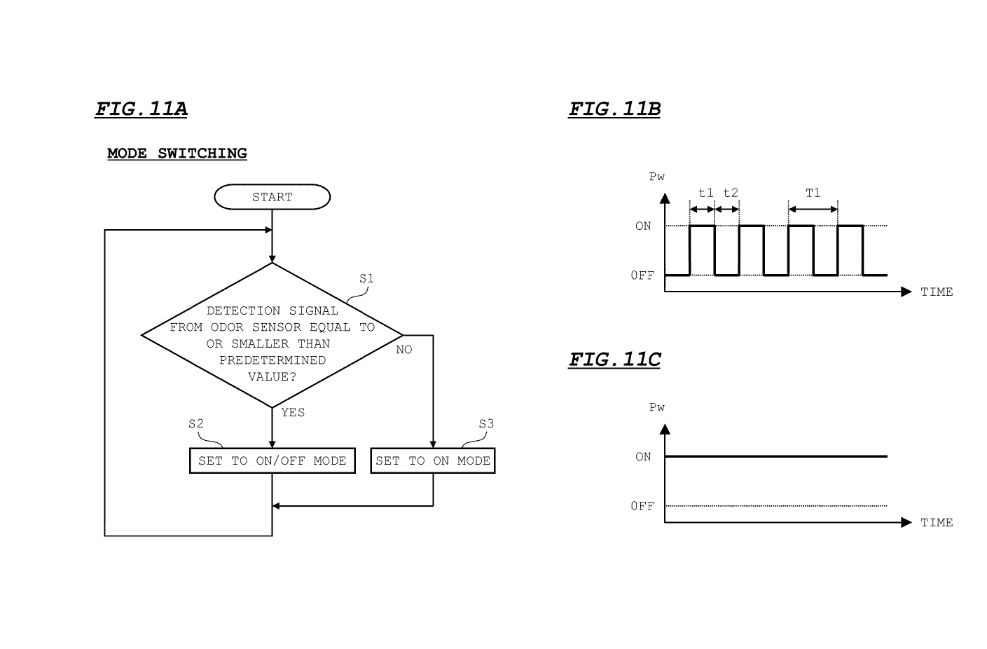

FIGS. 11A through 11C are diagrams showing a control method to be performed by the control circuit 170.

FIG. 11A is a flowchart showing mode switching of the deodorizing device 1.

During activation of the deodorizing device 1, the control circuit 170 determines whether a detection signal from the odor sensor 140 is equal to or smaller than a predetermined value (S1). In the case where the control circuit 170 determines that the detection signal from the odor sensor 140 is equal to or smaller than the predetermined value (S1: YES), the control circuit 170 sets an on-off control pattern of the LED 21 to “ON/OFF mode” (S2). In the case where the control circuit 170 determines that the detection signal from the odor sensor 140 is larger than the predetermined value (S1: NO), the control circuit 170 sets the on-off control pattern of the LED 21 to “ON mode” (S3). After the processings of Steps S2 and S3 are performed, the processing is returned to Step S1, and the sequence is repeated.

In the case where the determination result in Step S1 is negative, there is a case that the air contains a large amount of the material to be purified, and the photocatalysis films 13 may frequently contact the material to be purified due to diffusion of the material to be purified. In such a case, the on-off control pattern is set to “ON mode” for enhancing the purification performance by the photocatalysis films 13.

FIG. 11B is a diagram showing an on-off control pattern when the LED 21 is in “ON/OFF mode”.

When the on-off control pattern of the LED 21 is set to “ON/OFF mode”, as shown in FIG. 11B, the control circuit 170 controls the LED 21 so that the on-period is set to t1, the off-period is set to t2, and the on-off cycle is set to T1. The off period t2 is set to a time capable of sufficiently suppressing a temperature rise of the photocatalysis films 13 whose temperature has increased due to an on-state of the LED 21.

FIG. 11C is a diagram showing an on-off control pattern when the LED 21 is in “ON mode”.

When the on-off control pattern of the LED 21 is set to “ON mode”, as shown in FIG. 11C, the control circuit 170 controls the LED 21 to keep an on-state at a constant level.

In the case where the LED 21 is controlled as shown in FIGS. 11A through 11C, it is possible to securely purify the material to be purified, even in the case where the air to be fed to the purification region 110c contains only a trace amount of the material to be purified at the time of activation of the deodorizing device 1.

Specifically, if the LED 21 is turned on in a state that the air contains only a trace amount of the material to be purified, the temperature of the photocatalysis films 13 increases. As a result, the temperature of the air in the vicinity of the purification plates 10 increases, which makes it difficult to adhere, to the adsorption films 14, the material to be purified that is contained in the air in the purification region 110c. In the above configuration, however, the on-off control pattern of the LED 21 is set to “ON/OFF mode”, and a temperature rise of the photocatalysis films 13 is suppressed by causing the LED 21 to cyclically turn on and off at a predetermined time interval. Accordingly, the above configuration is advantageous in easily adhering the material to be purified to the adsorption films 14 to thereby easily purify the material to be purified.

As described above, in the deodorizing device 1 of the example, the material to be purified that is contained in the air drawn through the air intake port 110a is taken and decomposed by a photocatalytic action of the photocatalysis films 13 of the purification plates 10. The air that has been purified in the purification unit 100 is drawn out of the purification unit 100 through the air exhaust port 110b. In this way, the air in the vicinity of the deodorizing device 1 is purified.

Further, in the deodorizing device 1 of the example, in the case where the amount of the material to be purified is trace at the time of activation of the deodorizing device 1, the on-off control pattern of the LED 21 is set to “ON/OFF mode” to thereby suppress a temperature rise of the photocatalysis films 13. The above configuration is advantageous in efficiently adsorbing the trace amount of the material to be purified onto the purification plates 10 to thereby more securely purify the material to be purified.

Further, even in the case where the on-off control pattern of the LED 21 is set to any one of the aforementioned modes, as described in the example of the purification unit 100, the above configuration is more advantageous in securely purifying the material to be purified, because the purification performance of the purification plates 10 differ between the vicinity of the front plate 50 and the vicinity of the back plate 60 of the purification unit 100.

The on-off control pattern of the LED 21 shown in FIGS. 11A through 11C is switched between “ON/OFF mode” and “ON mode”. The invention is not limited to the above. Multiple on-off control patterns with each of which pulse emission having a different duty ratio is performed may be prepared, and these on-off control patterns may be switched as necessary.

Further, in the case where a semiconductor laser is used in the purification unit 100, in place of the LED 21, the on-off control pattern of the semiconductor laser is switched by the control circuit 170 substantially in the same manner as described above. In the above modification, multiple on-off control patterns of laser light to be emitted with different powers may be prepared, and these on-off control patterns may be switched as necessary.

Further, in the deodorizing device 1 of the example, the on-off control pattern of the LED 21 is automatically switched, based on a detection signal from the odor sensor 140. The invention is not limited to the above. A mode switching switch may be provided in the deodorizing device 1, and the user is allowed to manually switch the on-off control pattern.

The embodiment of the invention has been described as above. The invention is not limited to the foregoing embodiment, and the embodiment of the invention may be changed or modified in various ways other than the above.

For instance, in the purification unit of the example, UV-activated photocatalysis material is used. Alternatively, a conventional visible light-activated photocatalysis material may be used, in place of the UV-activated catalyst. The reason for using UV-activated photocatalysis material in the purification unit of the example is because the UV-activated photocatalysis material has enhanced purification performance. The purification performance of a conventional visible light-activated material is only about one-tenth of the purification performance of TiO₂ (anatase crystal) of UV activated type. However, as far as the visible light-activated material has a performance exceeding the performance of a UV-activated film, it is possible to select a light source most suitable for such a visible light-activated material and capable of generating a sufficient photocatalytic activity.

Further, in the example, as shown in FIG. 5, the photocatalysis film 13 of the purification plate 10 is configured to have a varied thickness in X-axis direction. Alternatively, the photocatalysis film 13 may be configured to have a constant thickness. In the above modification, since the light intensity on the region of the purification plate 10 corresponding to X-axis minus side is large with respect to the center of the purification plate 10. Accordingly, it is possible to enhance the purification performance of the purification plate 10 on X-axis minus side.

Further, in the example, as shown in FIG. 5, the transmissive films 12, the photocatalysis films 13, and the adsorption films 14 are laminated in a vertical direction with respect to the substrate 11 of the purification plate 10. The invention is not limited to the above. As shown in FIG. 1A, it is possible to laminate a transmissive film 12, a photocatalysis film 13, and an adsorption film 14 on one surface side of the substrate 11 of the purification plate 10.

Further, in the example of the purification unit 100, the LED 21 is used as a light source for causing a photocatalysis reaction. Alternatively, a semiconductor laser may be used in place of the LED 21. A semiconductor laser is a coherent light source, and is effective with respect to a specific crystal plane.

Further, in the example, the curved surface configurations of the reflection plates 30 through 33 are expressed by parabolic curves in X-Z plane. The invention is not limited to the above. Alternatively, the curved surface configuration may be expressed by another curve such as an elliptical curve. In the above modification, the LED 21 and the reflection plates 30 through 33 are adjusted in such a manner that the light intensity on the purification plates 10 is increased toward X-axis minus side on the purification plates 10.

Furthermore, in the example, the curved surface configuration of the reflection plate in the purification unit 100 is expressed by one parabolic curve or three parabolic curves. The invention is not limited to the above. Alternatively, the curved surface configuration may be expressed by two parabolic curves or more than three parabolic curves. In other words, two reflection plates or more than three reflection plates may be disposed in the purification unit 100.

Furthermore, LEDs may be additionally provided in the purification unit 100 configured based on FIG. 7A, as described in the following.

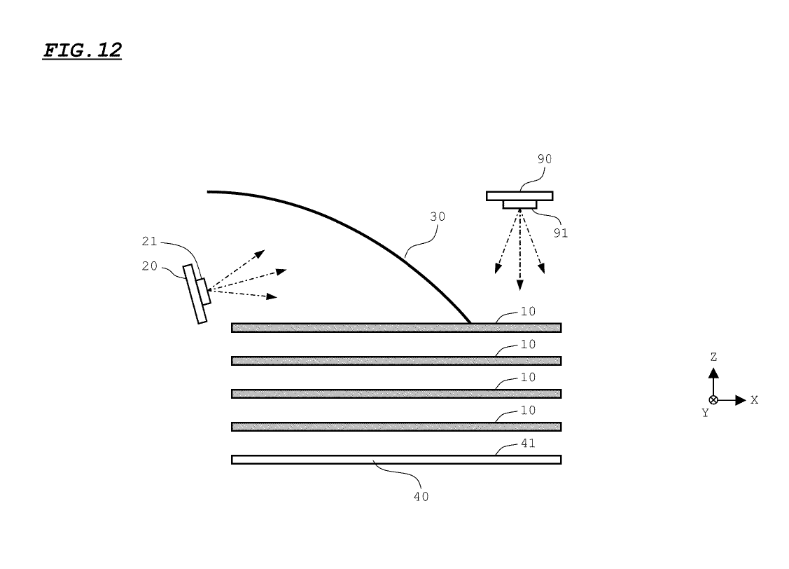

FIG. 12 is a side view of a purification unit 100 in which LEDs are additionally provided, when viewed from Y-axis direction.

As shown in FIG. 12, a light emitting unit 90 is disposed above a right-end region on a purification plate 10 closest to the light source in the purification unit 100. In the light emitting unit 90, plural LEDs 91 are disposed in Y-axis direction substantially in the same manner as the light emitting unit 20. The light emitting unit 90 causes each of the LEDs 91 to emit light, based on a control signal to be inputted to the light emitting unit 90 substantially in the same manner as the light emitting unit 20. The LED 91 emits light at a wavelength of 375 nm toward the purification plates 10. The LED 91 is disposed in such a manner that the optical axis of light to be emitted from the LED 91 perpendicularly intersects the purification plates 10.

In the above configuration, it is possible to individually control emissions of the LED 21 and the LED 91. Accordingly, it is possible to individually control a light intensity on a region corresponding to X-axis minus side and on a region corresponding to X-axis plus side of the purification plates 10.

In the case where the purification unit 100 configured as shown in FIG. 12 is incorporated in the deodorizing device 1, as shown in FIGS. 11A through 11C, the LED 91 is operable to switch the on-off control pattern by a detection signal from an odor sensor 140 substantially in the same manner as the LED 21. Alternatively, the LED 21 may be constantly set to “ON mode”, and the LED 91 may be operable to switch the on-off control pattern, as shown in FIGS. 11A through 11C.

The embodiment of the invention may be changed or modified in various ways as necessary, as far as such changes and modifications do not depart from the scope of the claims of the invention hereinafter defined.

What is claimed is:

1. A purification unit for purifying air by a photocatalysis reaction, comprising:
a light source which emits light;
a purification plate which causes the photocatalysis reaction by irradiation of the light; and
a reflection plate which has a curved surface configuration, and is configured to reflect the light emitted from the light source for guiding the reflected light to the purification plate, wherein
a layout of the light source and the curved surface configuration of the reflection plate are defined in such a manner that an irradiation intensity of the light on the purification plate is biased toward an upstream side of an air flow path in the purification unit.

2. The purification unit according to claim 1, wherein
the curved surface configuration of the reflection plate is expressed by one or more parabolic curves on a flat plane which is perpendicular to the purification plate and parallel to an air flow direction in the purification unit.

3. The purification unit according to claim 1, wherein
the purification plate has a photocatalysis film, and
a thickness of the photocatalysis film at an upstream of the air flow path in the purification unit is configured to be larger than a thickness of the photocatalysis film at a downstream of the air flow path in the purification unit.

4. The purification unit according to claim 3, wherein
the photocatalysis film of the purification plate is laminated on an upper surface side and on a lower surface side of the purification plate.

5. The purification unit according to claim 1, wherein
the purification plate is light transmissive, and a plurality of the purification plates are disposed at a predetermined interval in a direction perpendicular to a surface of the purification plate.

6. A deodorizing device, comprising:
a purification unit which purifies air by a photocatalysis reaction;
a fan which causes the air to flow into the deodorizing device; and
a controller which controls the fan and a light source in the purification unit, wherein
the purification unit is provided with:
the light source which emits light;
a purification plate which causes the photocatalysis reaction by irradiation of the light; and
a reflection plate which has a curved surface configuration, and is configured to reflect the light emitted from the light source for guiding the reflected light to the purification plate, and
a layout of the light source and the curved surface configuration of the reflection plate are defined in such a manner that an irradiation intensity of the light on the purification plate is biased toward an upstream side of an air flow path in the purification unit.

7. The deodorizing device according to claim 6, wherein
the curved surface configuration of the reflection plate is expressed by one or more parabolic curves on a flat plane which is perpendicular to the purification plate and parallel to an air flow direction in the purification unit.

8. The deodorizing device according to claim 6, wherein
the purification plate has a photocatalysis film, and
a thickness of the photocatalysis film at an upstream of the air flow path in the purification unit is configured to be larger than a thickness of the photocatalysis film at a downstream of the air flow path in the purification unit.

9. The deodorizing device according to claim 8, wherein
the photocatalysis film of the purification plate is laminated on an upper surface side and on a lower surface side of the purification plate.

10. The deodorizing device according to claim 6, wherein
the purification plate is light transmissive, and a plurality of the purification plates are disposed at a predetermined interval in a direction perpendicular to a surface of the purification plate.
